(19) United States
(12) Patent Application Publication
(10) Pub. No.: US 2019/0083391 A1
(43) Pub. Date: Mar. 21, 2019

(54) ORALLY DISINTEGRATING TABLETS FOR TREATMENT OF PEPTIC ULCER

(71) Applicant: Balto Therapeutics, New York, NY (US)

(72) Inventor: Courtney Oliver Bond, Mount Vernon, NY (US)

(21) Appl. No.: 15/811,072

(22) Filed: Nov. 13, 2017

Related U.S. Application Data
(60) Provisional application No. 62/559,740, filed on Sep. 18, 2017.

(51) Int. Cl. A61K 9/00; A61K 31/40; A61K 9/20

(52) U.S. Cl. CPC A61K 9/0056; A61K 31/40; A61K 9/2072; A61K 9/2018; A61K 9/2063

(57) ABSTRACT

An orally disintegrating tablet which comprises microscopic pores, at least one excipient and a therapeutically effective amount of glycopyrrolate for treating peptic ulcer. A method for making the tablets is provided as well.

### CROSS-REFERENCE TO RELATED APPLICATION

This application claims a benefit of priority to U.S. Provisional Patent Application 62/559,740 filed Sep. 18, 2017, the entire disclosure of which is incorporated herein by reference.

### TECHNICAL FIELD

The disclosure relates to orally disintegrating glycopyrrolate tablets for treatment of peptic ulcer and methods for treating patients with the formulations.

### BACKGROUND

Peptic ulcer is an open sore in the upper digestive tract. The sores can develop in the lining of a patient's stomach (gastric ulcer), small intestine (duodenal ulcer) and/or esophagus. Peptic ulcer is a very common condition in the United States with some reports suggesting that about 10% of the United States population develops a duodenal ulcer at some point in their lives. Often, the sores are painful. Other symptoms may include abdominal pain, nausea, vomiting loss of appetite and in some cases bleeding.

Various causes of peptic ulcer include excessive stomach acid production, a bacterial infection with a bacterium Helicobacter pyroli, radiation therapy and/or taking aspirin and/or nonsteroidal anti-inflammatory drugs (NSAIDs).

In order to control excessive stomach acid production in peptic ulcers, anticholinergic drug—glycopyrrolate can be used. However, not all patients have a good tolerance to an oral glycopyrrolate formulation which cannot be easily dissolved in the patient's saliva and may have to be swollen as an intact tablet. Some of the patients, including patients with dysphagia, cannot swallow a glycopyrrolate tablet. Oral formulations with orally disintegrating glycopyrrolate for treating sialorrhea are known from PCT/US2008/061025, but there is a need in the art for orally disintegrating glycopyrrolate formulations for treatment of peptic ulcer. U.S. Pat. No. 5,298,261 provides methods for preparing tablets which are vacuum-dried, but there is no indication in U.S. Pat. No. 5,298,261, that this method can be used with a quaternary amine compound without affecting the bioavailability of the compound.

### SUMMARY

This disclosure provides an orally disintegrating tablet comprising microscopic pores, at least one excipient and a therapeutically effective amount of glycopyrrolate for treating peptic ulcer. The orally disintegrating tablet may comprise the therapeutically effective amount of glycopyrrolate in the range from 0.5 mg to 5 mg. The peptic ulcer includes those which are accompanied by excessive stomach acid production. The excipient may be selected from a filler, binder and sugar. Glycopyrrolate can be a mixture of at least two stereoisomeric forms selected from R,R)-glycopyrrolate and (S,S)-glycopyrrolate, (R,S)-glycopyrrolate and (S,R)-glycopyrrolate. The tablets include those which orally disintegrate at least partially in less than 1 minute after being placed in a patient's mouth. The excipient can be selected from a filler, binder, sweetener, flavoring agent, diluent, disintegrant, lubricant, colorant, preservative and/or wetting agent. The excipient may comprise at least one of a filler, binder, or a sweetener.

In some formulations, the excipient comprises at least one or more from gelatin, mannitol and trehalose. In some formulations, the excipient may comprise trehalose, sucrose, glucose, saccharine, aspartame or any mixture thereof.

This disclosure also includes a method of treating a patient in need of treatment for peptic ulcer, the method comprising administering to the patient an orally disintegrating tablet comprising microscopic pores, at least one excipient and a therapeutically effective amount of glycopyrrolate for treating peptic ulcer. The patient is administered from 0.5 mg to 5 mg of glycopyrrolate from one to four times daily. This treatment can be beneficial to patients with duodenal ulcer. The orally disintegrating tablet of the disclosure dissolves or disintegrates rapidly with saliva, thus eliminating the need for chewing the tablet, swallowing an intact tablet, or taking the tablet with water. The formulations can be also used for treatment of other medical conditions where it is important to control excessive stomach acid production and/or reduce secretion in the mouth, throat and/or airways.

In further aspect, the disclosure provides a method of making an orally disintegrating tablet comprising microscopic pores, at least one excipient and a therapeutically effective amount of glycopyrrolate for treating peptic ulcer. In this method, a water-based suspension is prepared of glycopyrrolate in the amount from 0.5 mg to 5 mg per one dosage with at least one excipient. The suspension is dozed into molds and frozen. The frozen suspension is the dried under low pressure or in complete vacuum in order to sublimate water and create microscopic pores.

### DETAILED DESCRIPTION

This disclosure provides an orally disintegrating tablet comprising a therapeutically effective amount of glycopyrrolate for treating peptic ulcer. Glycopyrrolate also known as glycopyrronium bromide is a quaternary amine with the following formula:

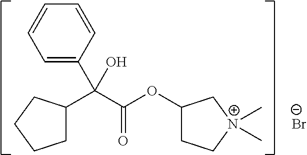

Glycopyrrolate exists in four distinct stereoisomeric forms: (R,R)-glycopyrrolate and (S,S)-glycopyrrolate, (R,S)-glycopyrrolate and (S,R)-glycopyrrolate. In some embodiments, a glycopyrrolate formulation of this disclosure comprises a mixture of at least two out of four stereoisomers: at least a mixture of (R,R)-glycopyrrolate and (S,S)-glycopyrrolate; at least a mixture of (R,R)-glycopyrrolate and (R,S)-glycopyrrolate or at least a mixture of (R,R)-glycopyrrolate and (S,R)-glycopyrrolate. In some embodiments, a glycopyrrolate formulation of this disclosure comprises a mixture of any three out of four stereoisomers. In some embodiments, a glycopyrrolate formulation comprises a mixture of all four stereoisomers. In other embodiments, the formulation comprises only one stereoisomer: (R,R)-glycopyrrolate, (S,S)-glycopyrrolate, (R,S)-glycopyrrolate or (S,R)-glycopyrrolate.

The term “therapeutically effective amount of glycopyrrolate for treating peptic ulcer” is to be understood broadly and means a dosage of glycopyrrolate effective to treat, ameliorate and/or improve symptoms of peptic ulcer in a patient. The therapeutically effective amount may be in the range from 0.5 mg to 5 mg per one oral tablet. The formulation can be taken from one to four times daily. Preferred formulations may comprise 1 mg, 2 mg or 3 mg of glycopyrrolate. The tablet can be administered in combination with other medications.

The glycopyrrolate tablet formulations of this disclosure disintegrate orally. These tablets dissolve or disintegrate rapidly when they are put in contact with the patient's saliva. Thus, there is no need for a patient to swallow an intact tablet or chew it. Instead, the tablet rapidly disintegrates in the patient's mouth.

Rapid oral disintegration means that a tablet breaks up into smaller pieces and/or the tablet becomes at least partially dissolved soon after the tablet is placed in the patient's mouth. In one preferred embodiment, the glycopyrrolate tablet formulations of the present disclosure are taken orally and they at least partially orally disintegrate in less than 1 minute, less than 50 seconds, less than 40 seconds or less than 30 seconds after being placed in the patient's mouth.

The glycopyrrolate tablet formulations of the present disclosure may comprise at least one excipient selected from a filler, binder, sweetener, flavoring agent, diluent, disintegrant, lubricant, colorant, preservative and/or wetting agent.

Some formulations comprise glycopyrrolate as an active ingredient in the amount from 0.5 mg to 5 mg and at least one of the following as an excipient: a filler, binder, sweetener and/or flavoring agent such as for example grape or cherry flavoring agent. A suitable sweetener may include, but is not limited to, at least one of the following various sugars (mono-, di-, and polysaccharides) such as for example, trehalose, sucrose, glucose, saccharine and aspartame. A suitable filler may include, but is not limited to, at least one of mannitol, sorbitol, and xylitol, or mixtures thereof. A suitable binder may include, but is not limited to, at least one of acacia, gelatin, pre-gelatinized starch, starch, methylcellulose, sodium carboxymethyl cellulose, ethyl cellulose, hydroxypropylmethyl cellulose, polyvinylpyrrolidone, and polyacrylamide.

Other ingredients in the glycopyrrolate tablet formulations of the present disclosure may include at least one of the following: a diluent, disintegrant, lubricant, colorant, preservative and/or wetting agent.

An orally disintegrating tablet comprising a therapeutically effective amount of glycopyrrolate for treating peptic ulcer is made by lyophilization (freeze-drying). Thus, the invention provides an orally disintegrating freeze-dried tablet comprising a therapeutically effective amount of glycopyrrolate for treating peptic ulcer. In some freeze-drying methods, a suspension comprising a therapeutically effective amount of glycopyrrolate for treating peptic ulcer is made with suitable excipients and water. The suspension is then aliquoted into molds. The aliquots are caused to freeze and exposed to drying under low pressure. The drying causes frozen water to evaporate from the solid phase to the gas phase and creates microscopic pores in a tablet formulation. The step of freezing may be performed at a temperature in the range from −50° C. to −80° C. The orally disintegrating freeze-dried tablet comprises microscopic pores and comprises a therapeutically effective amount of glycopyrrolate for treating peptic ulcer.

To make an oral disintegrating tablet comprising a therapeutically effective amount of glycopyrrolate for treating peptic ulcer, glycopyrrolate is combined with a suitable excipient which may comprise pharmaceutically compatible materials which may include a filler, which can be mannitol, and a binder, which can be gelatin, and a sweetener, which can be trehalose. The mixture is then either dissolved or dispersed in water. This can be accomplished by using a mixer. Additional ingredients, such as a flavoring agent, diluent, disintegrant, lubricant, colorant, preservative and/or wetting agent can be also added.

The resulting suspension may be dosed by weight into individual pre-formed blister molds in a fully automated continuous filling process. It is important that the suspension remains homogeneous during dosing. This process is regulated and monitored to ensure that each dose contains the exact amount of active ingredient—glycopyrrolate.

Once filled, the blister molds are passed through a freezing tunnel cooled by liquid nitrogen. This freezes water in the glycopyrrolate suspension and makes it ready for loading into low-temperature storage ahead of a freeze-drying process. As soon as a sufficient number of blister molds have been filled and frozen, lyophilization can begin. The blister molds are then transferred to a freeze-dryer. When the lyophilization is complete, the blister molds are passed through a blister sealer, where they are sealed with aluminium foil or a suitable paper laminate. The sheets of blister molds are cut to size, and the foil perforated to facilitate opening by the patient.

What is claimed is:

1. An orally disintegrating tablet which comprises microscopic pores, at least one excipient and a therapeutically effective amount of glycopyrrolate for treating peptic ulcer.

2. The orally disintegrating tablet of claim 1, wherein the therapeutically effective amount of glycopyrrolate is in the range from 0.5 mg to 5 mg.

3. The orally disintegrating tablet of claim 1, wherein the peptic ulcer is accompanied by excessive stomach acid production.

4. The orally disintegrating tablet of claim 1, wherein the excipient is selected from a filler, binder and sugar.

5. The orally disintegrating tablet of claim 1, wherein glycopyrrolate is a mixture of at least two stereoisomeric forms selected from R,R)-glycopyrrolate and (S,S)-glycopyrrolate, (R,S)-glycopyrrolate and (S,R)-glycopyrrolate.

6. The orally disintegrating tablet of claim 1, wherein the tablet orally disintegrates at least partially in less than 1 minute after being placed in a patient's mouth.

7. The orally disintegrating tablet of claim 1, wherein the excipient selected from a filler, binder, sweetener, flavoring agent, diluent, disintegrant, lubricant, colorant, preservative and/or wetting agent.

8. The orally disintegrating tablet of claim 1, wherein the excipient comprises at least one of a filler, binder, or a sweetener.

9. The orally disintegrating tablet of claim 1, wherein the excipient comprises at least one or more from gelatin, mannitol and trehalose.

10. The orally disintegrating tablet of claim 1, wherein the excipient comprises trehalose, sucrose, glucose, saccharine, aspartame or any mixture thereof.

11. A method of treating a patient in need of treatment for peptic ulcer, the method comprising administering to the patient an orally disintegrating tablet comprising microscopic pores, at least one excipient and a therapeutically effective amount of glycopyrrolate for treating peptic ulcer.

12. The method of claim 11, wherein the patient is administered from 0.5 mg to 5 mg of glycopyrrolate from one to four times daily.

13. The method of claim 11, wherein the peptic ulcer is duodenal ulcer.

14. A method of making an orally disintegrating tablet comprising microscopic pores, at least one excipient and a therapeutically effective amount of glycopyrrolate for treating peptic ulcer, the method comprising:
preparing a water-based suspension of glycopyrrolate in the amount from 0.5 mg to 5 mg per one dosage with at least one excipient;
dosing the suspension into molds;
freezing the suspension in the molds; and
drying the frozen suspension, and thereby obtaining an orally disintegrating tablet comprising microscopic pores, at least one excipient and a therapeutically effective amount of glycopyrrolate for treating peptic ulcer.

15. The method of claim 14, wherein the excipient is selected from a filler, binder, sweetener, flavoring agent, diluent, disintegrant, lubricant, colorant, preservative and/or wetting agent.

16. The method of claim 14, wherein the excipient comprises at least one of a filler, binder, or a sweetener.

17. The method of claim 14, wherein the excipient comprises at least one or more from gelatin, mannitol and trehalose.

18. The method of claim 14, wherein the excipient comprises trehalose, sucrose, glucose, saccharine, aspartame or any mixture thereof.
